(19) United States
(12) Patent Application Publication
(10) Pub. No.: US 2006/0073589 A1
(43) Pub. Date: Apr. 6, 2006

(54) Rapid generation of activated mononuclear antigen presenting cells from monocytes

(76) Inventors: Filippo Belardelli, Roma (IT); Tiziana Di Pucchio, Roma (IT); Stefano Maria Santini, Roma (IT); Caterina Lapenta, Firenze (IT); Mariantonia Logozzi, Roma (IT); Stefania Parlato, Roma (IT)

Correspondence Address: YOUNG & THOMPSON, 745 SOUTH 23RD STREET, 2ND FLOOR, ARLINGTON, VA, 22202, US

(21) Appl. No.: 10/511,748

(22) PCT Filed: Apr. 15, 2003
(86) PCT No.: PCT/EP03/03922

§ 371 Date: Sep. 6, 2005

(30) Foreign Application Priority Data
Apr. 19, 2002 (EP) 02290994.9

(51) Int. Cl. A61K 35/14; C12N 5/08

(52) U.S. Cl. 435/372; 435/375; 424/93.7

(57) ABSTRACT

A cell population prepared from blood mononuclear cells, the mononuclear cells being peripheral blood mononuclear cells (PBMC) or CD 14+ monocytes, the population containing from about 10% to about 50% of activated mononuclear antigen presenting cells (APCs) having the following characteristics: • they express surface markers CD2, CD83 and CD14, MHC class I and MHC class II molecules • they secrete TNF-α • they are able to stimulate allogenic T lymphocytes, as shown by MLR measurements • they are able to stimulate autologous T lymphocytes proliferation in an antigen specific way.

The present invention relates to new activated mononuclear antigen-presenting cells, means processes and kit for preparing the same, and their use for the preparation of pharmaceutical compounds.

Dendritic cells (DCs) are professional antigen-presenting cells, derived from hematopoietic progenitor cells, playing a central role in the initiation of the immune response to pathogens (1-3). The recent progress on DC biology has revealed the heterogeneity of these cells and various types of DCs endowed with different phenotypic and functional properties have been characterized under both in vivo and in vitro conditions (4). However, the mechanisms regulating generation, functions and survival of DCs in response to infections or exposure to foreign or modified antigens are largely unknown.

Upon exposure to pathogens and other danger signals, DCs undergo a complex maturation process, characterized by the expression of a distinct pattern of membrane molecules, which parallels the acquisition of high competence for antigen presentation and migration to the lymphoid organs (3). One typical marker of DC maturation is CD83, a 45 kD glycoprotein belonging to the Ig superfamily, predominantly expressed in vivo on the surface of some DC cell subsets, including skin Langherans cells and interdigitating reticulum cells present in the T cell zones of lymphoid organs (5). Although the physiological function of CD83 is still unknown, an association with the generation of highly active DCs is presumed, as it may play an important role in Ag presentation and/or cellular interactions (6).

Monocytes are highly versatile cells playing crucial roles in the maintenance of immune homeostasis. These cells circulate in the bloodstream, transmigrate through vascular endothelium and localize in peripheral tissues, where they mature into different types of tissue histiocytes or other cell types. In vitro studies have shown that monocytes can be the precursors of a particular subset of myeloid DCs (generally named as DC1), whose generation requires several days of culture in the presence of cytokines, followed by a further incubation step necessary to induce DC maturation (2, 4). In particular, even though some authors reported the appearance of a variable percentage of CD83⁺ DCs after a single step treatment of monocytes, either after transmigration across an endothelial barrier (7) or after a 3 day exposure to GM-CSF and type I interferon (8), it is generally thought that the expression of CD83 on monocyte-derived DCs requires two sequential events: i) the induction of DC differentiation from monocytes, after exposure for several days to GM-CSF and IL-4 (9); ii) induction of maturation, characterized by CD83 expression, after the subsequent addition of stimuli such as LPS, CD40L, virus infection or other factors (2). Although this method for preparing monocyte-derived DCs has allowed to carry out important studies on the biology of DCs, the physiological relevance of this pattern of DC differentiation is questionable, especially because exposure of monocytes to IL-4 can hardly mimic the cytokine milieu likely to be present at the infection site.

Distinct subtypes of human circulating DCs have been detected in the blood. In particular, Zhou and Tedder had described a rare population of human blood DCs selectively expressing CD83 (10). Moreover, circulating CD2⁺ monocytes have been considered as DC precursors (11) and as a special subset of peripheral blood DCs (12). However, no relationship was established between CD2 and CD83 expression and freshly isolated CD2⁺ monocytes were described as CD83-negative cells (12).

The inventors report the unexpected finding that membrane CD83 expression is induced, by post-transcriptional mechanisms, in CD2⁺/CD14⁺ monocytes (i.e. cells expressing both markers) soon after cell seeding, concomitantly with the acquisition of marked DC activities. IFN-α, but not IL-4, favors the persistence of membrane CD83 expression during culture time and rapidly renders these cells highly competent for inducing a primary T cell response.

Advantages of the invention are given in that it describes a new cell population containing functional activated mononuclear antigen presenting cells (APCs). Said population is obtainable thereby from freshly isolated PBMCs or monocytes after a single step treatment. The invention also provides a particularly rapid procedure for the production of these new APCs. This procedure may furthermore be carried out without any ex vivo culture step, in a close single use system, therefore avoiding possible contaminations and limiting manipulations and transports.

The present invention relates to a cell population prepared from blood mononuclear cells, said mononuclear cells being peripheral blood mononuclear cells (PBMC) or CD14+ monocytes, said population containing from about 5% to about 50% of activated mononuclear antigen presenting cells having the following characteristics:
they express surface markers CD2, CD83 and CD14, MHC class I and MHC class II molecules
they secrete TNF-α
they are able to stimulate allogenic T lymphocytes, as shown by MLR measurements.

Mononuclear cells particularly preferred for the preparation of cells according to the invention are possibly total unseparated peripheral blood mononuclear cells (PBMC), isolated from blood by any process known by a person skilled in the art, such as apheresis. Total PBMCs contain mainly monocytes, lymphocytes and Natural Killer (NK) cells. As an alternative, monocytes may be isolated from blood, for example by density gradient centrifugation, adherence or positive immunoselection using anti-CD14 conjugated magnetic beads. These monocytes isolated from blood are designated as CD14⁺ monocytes. An important fraction of this CD14+ monocytes population also expresses CD2 marker on its surface and are then designated as CD14+/CD2+ monocytes.

By “antigen-presenting cells” (APCs) is meant cells able to present antigens to T lymphocytes and to stimulate their proliferation. By “activated mononuclear APCs” are defined mononuclear cells characterized by the membranar or extracellular expression of proteins which were not synthesised or not exported by non-activated mononuclear cells, but the corresponding mRNA of which were already present in non-activated mononuclear cells, therefore allowing protein expression within a few hours process. Activated mononuclear APCs also exhibit immunostimulating properties not presented by mononuclear cells. However, activated mononuclear APCs do not exhibit phenotypic or functional characters that are associated to cells differentiated from monocytes after several days of culture, such as the presence of dendrites, for monocyte-derived dendritic cells, or the expression of all co-stimulatory molecules, such as CD80. An other difference between activated mononuclear cells and differentiated monocytes resides in the high proportion of cells positive for the CD14 surface marker for the first ones.

Activated mononuclear APCs from a cell population according to the invention are better stimulators of T and B cell response than freshly purified monocytes. Therefore, such a cell population is suitable for the generation of an immune response in a patient in need thereof. Also, activated mononuclear APCs exhibit an immunophenotype (including receptors for monokines and chemokines), morphology, and immunological activity not present in dendritic cells (DCs), and particularly not in monocyte-derived DCs differentiated according to processes well known in the art (1, 2, 3).

In a particular embodiment, the invention relates to a cell population prepared from blood mononuclear cells containing from about 5% to about 50% of activated mononuclear antigen presenting cells having the following characteristics:
they express surface markers CD2, CD83 and CD14, MHC class I and MHC class II molecules
they secrete TNF-α
they are able to stimulate allogenic T lymphocytes, as shown by MLR measurements.
they are able to stimulate autologous T lymphocytes proliferation in the presence of a specific antigen

In a particular embodiment, the present invention relates to a cell population prepared from blood mononuclear cells, said population containing from about 10% to about 50% of activated mononuclear antigen presenting cells such as above described.

If necessary, activated mononuclear APCs may be isolated from the cell population according to the invention by means such as elutriation and/or negative selection from lymphocytes on magnetic beads, for example on magnetic beads coated with anti-CD3 antibodies.

The present invention also relates to activated mononuclear APCs prepared from blood mononuclear cells and having the following characteristics:
they express surface markers CD2, CD83 and CD14, MHC class I and MHC class II molecules
they secrete more than 50 pg/ml of TNF-α
they are able to stimulate allogenic T lymphocytes, as shown by MLR measurements.

In a particular embodiment, the present invention relates to activated mononuclear APCs prepared from blood mononuclear cells and having the following characteristics:
they express surface markers CD2, CD83 and CD 14, MHC class I and MHC class II molecules
they secrete more than 50 pg/ml of TNF-α
they are able to stimulate allogenic T lymphocytes, as shown by MLR measurements
they are able to stimulate autologous T lymphocytes proliferation in the presence of a specific antigen.

In another particular embodiment, the invention relates to activated mononuclear APCs such as above described, which express the following surface markers: CD2, CD14, CD83, CD54, CD58, CD86, MHC class I and MHC class II molecules.

In another particular embodiment, the present invention relates to activated mononuclear antigen presenting cells possessing phagocytic properties, as shown by dextran-uptake capability. This potentiality is of particular interest for the intracellular accumulation of antigens to be processed and presented to the cell membrane.

In a particular embodiment, activated mononuclear APCs according to the invention do not secrete detectable levels of IL-10 and secrete less than 100 pg/ml of IL-12. By “secretion of IL-12” is meant “secretion of IL-12p70”. In this respect, they differ from dendritic cells that may secrete high levels of IL-12p70.

In an other particular embodiment, activated mononuclear APCs according to the invention are loaded with antigenic peptides or proteins, with a cellular extract containing at least one antigen or with nucleic acid molecules. Antigens against which the stimulation of an immune response is of interest are, for example, tumor-associated antigens, such as Melan-MART or MAGE, antigens overexpressed on tumors, such as Her-2/neu, MUC-1 or p53, or viral tumorigenic antigens, originating for example from Hepatitis C Virus, Human Papilloma Virus or Human T Lymphotropic Virus. They also can be infectious antigens from viral agents such as Human Immunodeficiency Virus, Hepatitis B Virus, Hepatitis C Virus, or other viruses. A cellular extract may be composed with extracts from one or several different types or lineages of cells. Activated mononuclear APCs according to the invention may also be loaded with pan carcinoma antigens such as peptides or polypeptides from human inducible hsp70.

The present invention also relates to the use of ligands having receptors on the surface of blood monocytes, of inducers of interferon synthesis by blood mononuclear cells or of a physical stress, as means allowing the preparation from blood mononuclear cells in an appropriate medium and for one to five hours, and preferably for less than four hours, of a cell population or of activated mononuclear APCs such as above described, said means being applied to the mononuclear cells from the initial stage of the preparation.

By “ligand having receptors on the surface of blood monocytes” is meant any ligand binding with high affinity to membrane receptors of monocytes and having transactivating properties.

Such ligands usable according to the invention are chosen among the group consisting of: cell-growth factors, complement polypeptides, muramyl dipeptide analogues, natural and synthetic endotoxin derivatives such as synthetic LipoPolySaccharides (LPS) analogues derived from Escherichia Coli or monophosphoryl lipid A, histamine, Vitamin D3, arachidonic acid metabolites, amino sulfonic acid derivatives, Bacillus Calmette-Guerin (BCG), bacterial membrane extracts such as FMKp (Fraction Membranaire Klebsiella pneumoniae) such as described in WO/0054790, or Ribomunyl®. The ligands of this group are generally considered as inducers of interferon synthesis by blood mononuclear cells.

In a more particular embodiment, detoxidied endotoxin derivatives are used at concentrations superior to acceptable contaminating endotoxin level for a culture medium, which is for example from about 0.001 to about 0.5 Endotoxin Units EU)/ml for AIM-V (Gibco-Life Technologies) culture medium. As an example, a detoxified endotoxin derivative such as monophosphoryl lipid A can be used at concentrations of about 10 μg/ml.

In another particular embodiment, the present invention relates to the use of cytokine having receptors on the surface of blood monocytes as a mean allowing the preparation from blood mononuclear cells in an appropriate medium and for one to five hours, and preferably for less than four hours, of a cell population or of activated mononuclear APCs such as above described, said mean being applied to the mononuclear cells from the initial stage of the preparation.

According to a particular embodiment of the invention, said cytokine having receptors on the surface of blood monocytes are chosen among a group consisting of: type I interferon (IFN), IFN-gamma, IL-12, IL-13, IL-18 and Granulocyte/Monocyte-Colony Stimulating Factor (GM-CSF). According to a more particular embodiment of the invention, said type I IFN is selected from the group consisting of any natural IFNα, any recombinant species of IFNα, natural or recombinant IFNβ and any synthetic type I IFN. In a further embodiment of the invention, said type I IFN concentration in the medium is in a range of about 100 to about 100,000 IU/ml, and preferably in a range of about 1,000 to about 10,000 IU/ml.

According to a particular embodiment of the invention, said cytokine is used at a concentration in the medium ranging from about 0.01 to about 10 μg/ml and preferably from about 0.1 to about 1 μg/ml.

Means such as above described may be used alone or in combination. In a preferred embodiment of the invention, type-I Interferon or IL-13 are used in combination with GM-CSF. In a preferred embodiment, type I IFN is used at a concentration of about 10,000 IU/ml, in combination with GM-CSF at a concentration of about 500 IU/ml.

The present invention also relates to the use of a physical stress as means for the generation, of activated mononuclear APCs. Said physical stress may be chosen among a group consisting of one of the following events: the separation of blood mononuclear cells from the plasma contained in the blood initially containing the mononuclear cells, the exposure of the blood mononuclear cells to a temperature variation of +/−3 to 8° C. from 37° C., to an osmotic change or to an electrical field.

Another aspect of the present invention relates to a process for preparing, from blood mononuclear cells, a cell population or activated mononuclear APCs such as above described, comprising a step of contacting said mononuclear cells with an appropriate medium for about one to about five hours and preferably for less than about four hours.

By “medium” is meant any medium appropriate for the cells survival or culture and differing from blood. Any medium suitable for ex vivo culturing of mononuclear cells is suitable. However, in the specific case of further use of the cells for human patients, culture media like AIM-V (Gibco-Life Technologies) or X-VIVO 20 are preferably used.

In a particular embodiment, the process of the invention is characterized in that said medium contains, from the initial stage of the preparation, a component selected from the group consisting of: ligands having receptors on the surface of blood monocytes.

By “initial stage of the preparation” is defined the moment where mononuclear cells are put in contact with the medium.

By “ligand having receptors on the surface of blood monocytes” is meant any ligand binding with high affinity to membrane receptors of monocytes and having transactivating properties. Such ligands are chosen among the group consisting of: cell-growth factors, complement polypeptides, muramyl dipeptide analogues, natural and synthetic endotoxin derivatives such as synthetic LipoPolySaccharides (LPS) analogues derived from Escherichia Coli or monophosphoryl lipid A, histamine, Vitamin D3, arachidonic acid metabolites, amino sulfonic acid derivatives, Bacillus Calmette-Guérin (BCG), bacterial membrane extracts such as FMKp (Fraction Membranaire Klebsiella pneumoniae) or Ribomunyl®. The ligands of this group are generally considered as inducers of interferon synthesis by blood mononuclear cells.

In a more particular embodiment, the process of the invention is characterized in that said medium contains, from the initial stage of the preparation, a component selected from the group consisting of: cytokine having receptors on the surface of blood monocytes and inducers of interferon synthesis by blood mononuclear cells.

By “cytokine having receptors on the surface of blood monocytes” is meant any cytokine binding with high affinity to membrane receptors of monocytes and having transactivating properties. Said definition includes cytokines such as interferons (IFN), such as type I IFN or IFN-gamma, IL-12, IL13, IL-18, and Granulocyte/monocyte-colony stimulating factor (GM-CSF), TNFα and TGFβ.

According to a particular embodiment of the invention, said cytokine is type I IFN and is selected from the group consisting of any natural IFNα, any recombinant species of IFNα, natural or recombinant IFNβ and any synthetic type I IFN. In a further embodiment of the invention, said type I IFN concentration in the medium is in a range of about 100 to about 100,000 IU/ml, and preferably in a range of about 1,000 to about 10,000 IU/ml.

Using the latter range and concentration, activated mononuclear APCs acquire the optimal expression of membrane markers associated with functional activity, with minimal toxic effects and good cell viability.

In another particular embodiment, said cytokine is chosen among the group consisting of: IL-12, IL-13, IL-18 or GM-CSF, said cytokines being present in the medium at a concentration ranging from about 0.01 to about 10 μg/ml and preferably from about 0.1 to about 1 μg/ml.

The concentration of GM-CSF, which promotes monocyte survival in culture, is generally expressed as IU/ml. In a process according to the invention, it is preferably form about 100 to about 2000 IU/ml, and is preferably of about 500 IU/ml. About the equivalence between IU and SI units, for GM-CSF “Leucomax” (Novartis), 1.67 10⁶ IU correspond to 150 μg of product.

In another particular embodiment, interferon or cytokines, such as above described, may be used in combination. In a preferred embodiment, the medium contains type I IFN, at a concentration of about 10,000 IU/ml, and GM-CSF, at a concentration of about 500 IU/ml.

According to another embodiment of the invention, a process for preparing a cell population or activated mononuclear APCs such as above described may comprise a step of exposing said blood mononuclear cells to a physical stress, which can be: the separation of blood mononuclear cells from the plasma contained in the blood initially containing the mononuclear cells, the exposure of the cells to a temperature variation of +/−3 to 8° C. from 37° C., to an osmotic change or to an electrical field. The “intensity” of such physical stress applied will be chosen so as to preserve viability and functionality of the mononuclear cells.

According to another embodiment, a process of the invention may be carried out by adding the medium with antigenic peptides or proteins, with a cellular extract containing at least one antigen or with nucleic acid molecules preferably encoding for antigens against which it is desirable to induce an immune response. Said added components are intended to be loaded onto the mononuclear cells. Cells may be loaded by direct adsorption of peptides on MHC molecules expressed on the surface of the cells, by phagocytosis of particulate antigens, pinocytosis of soluble antigens, affinity binding, fusion, intracellular nucleic acid (DNA or RNA) transfer, receptor mediated uptake or viral transfection according to methods known by a person skilled in the art.

The expression “inducers of interferon synthesis by blood mononuclear cells” relates to agents such as double stranded nucleic acids, to viral extracts, or to any other agent having such property known by a person skilled in the art.

In a further preferred embodiment, the process of the invention comprises:
a first step of contacting mononuclear cells with an appropriate medium for about one to about five hours and preferably for less than about four hours, for the preparation of activated mononuclear APCs
a second step of contacting said activated mononuclear APCs with a maturation agent.

Such an embodiment can be particularly suitable in all the cases, which can be identified by a skilled person, wherein a further maturation of the APCs obtained from the mononuclear cells as reported above is desired. By “maturation agent” is meant any means known as to induce the maturation of dendritic cells resulting into phenotypic and functional modification of the cells (13). Said agent may be chosen, for example, among bacterial membrane extracts, such as poly IC, IFN gamma, CD40 ligand, or anti-CD40 antibody, peptides or proteins, such as OmpA (14), or other agents such as the clinically approved Broncho-Vaxom oral vaccine (15).

Accordingly, a process for the preparation of activated mononuclear APCs comprises a step of washing said activated mononuclear APCs, so as to eliminate possible residues of agents used for the preparation or the maturation of the cells. If activated mononuclear APCs of the invention are prepared directly from an apheresis product and in connection with an apheresis machine, washings may be easily carried out by said apheresis machine.

The invention also relates to activated mononuclear antigen presenting cells such as obtained by a process such as above described. The present invention also relates to activated mononuclear APCs kept under a frozen form, in an appropriate cryopreservative solution until reinjection to a patient. A part of the activated mononuclear APCs of the invention can be taken from the close system in which they are prepared and kept frozen, so as to be used for a later injection. Cells may be diluted in an appropriate medium, such as one containing 10% of autologous serum +10% of dimethylsulfoxyde in a phosphate buffer saline. Cells according to the invention may also be conserved in a dehydrated form.

The invention also relates to a cell population or activated mononuclear APCs such as above described, or a composition comprising these cells, for use as a medicament.

The present invention further relates to a kit for preparing activated mononuclear APCs from blood mononuclear cells, in a close system allowing the exclusion of any conventional ex-vivo culture step, and comprising at least:
single use elements necessary for the activation process and the washings, including bag(s), appropriate medium, buffers and connecting tube(s), including connecting tube(s) to the apheresis machine.
possibly a composition comprising type I IFN and compatible additives,
possibly a composition comprising a cytokine and compatible additives,
possibly a composition comprising a cell growth factor and compatible additives,
possibly a composition comprising a ligand having receptors on the surface of blood monocytes and compatible additives,
possibly antigens to which an immune response is of interest.

This kit contains means for the reduction to practice of the process described in the present application. A skilled person can easily identify the additives suitable in the compositions reported above.

A kit according to the invention may also possibly include:
means for recovering and centrifuging blood to obtain a leucocyte concentrate,
means for the recovery of mononuclear cells from PBMC;
means for recovering lymphocytes and monocytes from other white cells and contaminating red cells,
means for the transfection of cells and for targeting antigens to blood mononuclear cells or to the activated mononuclear-APCs.
means for cell conservation, including for example glycerol or dimethylsulfoxyde.

A description of the invention is also given with the help of the annexed FIG. 7.

A further object of the present invention is a pharmaceutical composition or a vaccine comprising, as active principle, activated mononuclear APCs such as described above, together with a pharmaceutically acceptable carrier vehicle or an auxiliary agent, in a amount of about 10⁵ to about 10¹⁰, and preferably about 10⁷ to about 10⁸ of said cells per dose administered.

A further object of the present invention is a pharmaceutical composition or a cellular vaccine containing, as an adjuvant of an active principle, activated mononuclear APCs such as above described, in a amount of about 10⁵ to about 10¹⁰, and preferably about 10⁷ to about 10⁸ of said cells per dose administered.

Due to the properties of activated mononuclear APCs according to the invention and depending on the component they are associated with, said cells may be used as an active principle or as an adjuvant. Any vehicle, carrier, auxiliary agent and formulation adopted in art for manufacturing vaccines can be used in the vaccine of the invention. A skilled person can identify said components and all the steps of the relevant process of manufacturing.

The present invention also relates to the use of a cell population or of activated mononuclear APCs such as above described for the manufacture of a medicament for treating an infectious or neoplastic disease. In a particular embodiment, said pathology is a tumor-associated disease.

The present invention also relates to a method for prophylaxis and/or therapy of pathologies associated with the presence of an antigen in the human body and comprising a step of administering a vaccine including an immunogen for said antigen and activated mononuclear APCs of the invention to a subject in need thereof. Such an antigen, including viral, bacterial and tumor antigens such as above described, can be any molecule which presence is associated with a pathology. The present invention particularly relates to a method for prophylaxis and/or therapy of neoplastic and of infectious pathologies. Particular indications for neoplastic diseases are melanoma, prostate cancer, bladder cancer, breast cancer, blood malignancies and colorectal cancer. Anti-infectious pathologies particularly concerned are caused by Human Immunodeficiency virus, Hepatitis B and Hepatitis C virus.

The activated mononuclear APCs of the invention can be injected with specific antigens into a subject in need thereof, so that antigens are locally acquired by activated mononuclear APCs. The administration of activated mononuclear APCs of the invention is preferably intravenous in the peripheral circulation, at the end of the apheresis process, the patients possibly remaining connected to the machine during the whole process, or can also be directed at the site of the infection or within the primary tumor, metastases or draining lymph nodes.

In a particular embodiment, blood mononuclear cells are collected from patients previously mobilized, by the administration of GM-CSF or Flt-3 ligand, so as to increase the amount and proportion of myeloid, monocytes and precursor cells in peripheral blood. This allows to perform a shorter collection of mononuclear cells, because of the processing of a smaller volume of treated blood and to focus the treatment on the ex vivo activation process.

The process according to the invention, using a close system for antigenic activation of APCs without any transfer of the cells to dedicated clean rooms and incubators, is of particular interest for patients with viral pathologies, such as HIV, HBV, HCV. These patients are usually not included in adoptive therapies for regulatory and security reasons. The process and kit of the invention allow the activation of the mononuclear cells and the loading of the antigens for which the induction of an immune response is of therapeutic interest, during the apheresis process, ensuring optimal safety and efficacy.

### LEGENDS OF THE FIGURES AND TABLE

FIGS. 1A and 1B: In vitro up-regulation of CD83 expression on the cell membrane of freshly isolated CD14⁺ monocytes: correlation with the CD2 expression.

FIG. 1A shows flow cytometric analysis showing CD83 and CD2 expression in freshly isolated monocytes (“0 hours”, up and low left panels) versus monocytes cultured for 4 h in medium alone (“4 hours”, up and low right panels). The left and right upper panels show the surface staining for CD83-PE (y axis) versus CD14-FITC (x axis), whereas left and right lower panels show the surface staining CD83-PE (y axis) and anti-CD2-FITC (x axis). Isotype control antibodies were used to set negativity limits according to their location. Similar profiles were obtained with monocytes from 10 different donors.

FIG. 1B puts in evidence the linear relationship existing between the percentage of starting CD2⁺/CD14⁺ monocytes (x-axis) and the percentage of monocyte-derived CD83⁺ cells (y-axis) recovered after 4 h of culture. The correlation was evaluated by regression analysis (the regression coefficient is 0.633) using data obtained with monocytes from 18 donors.

FIGS. 2A, 2B and 2C: Post-transcriptional up-regulation of CD83 expression on the cell surface of cultured monocytes and correlation with acquisition of a high MLR activity.

FIG. 2A shows the CD83 expression of isolated monocytes cultured in medium alone (“medium”, up and left panel) or in medium containing actinomycin D 100 ng/ml (“+ActD”, up and right), cyclohexamide 100 μM (“Chx”; low and left) or brefeldin A 5 μg/ml (“BfA”; low and right) for 4 h. The dotted line indicates the FACS profile of cells stained with isotype control mAb. The data are representative of three independent experiments.

FIG. 2B represents the RT-PCR analysis of CD83 mRNA in freshly purified monocytes (time 0), monocyte-derived cells harvested at 4 h of culture, monocyte-derived immature and mature dendritic cells, respectively i-DCs and m-DCs. Negative control (CTR-) is represented by RNA extracted from a T cell line (CEM).

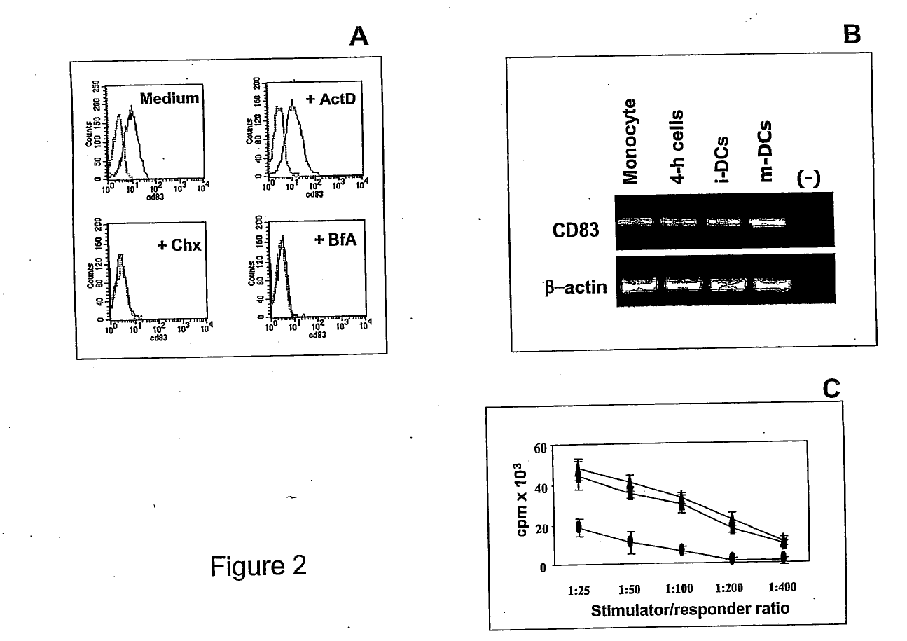

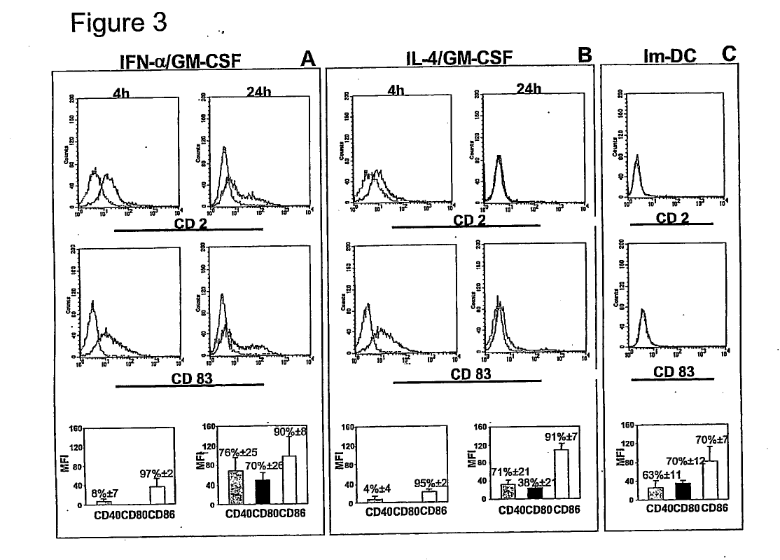

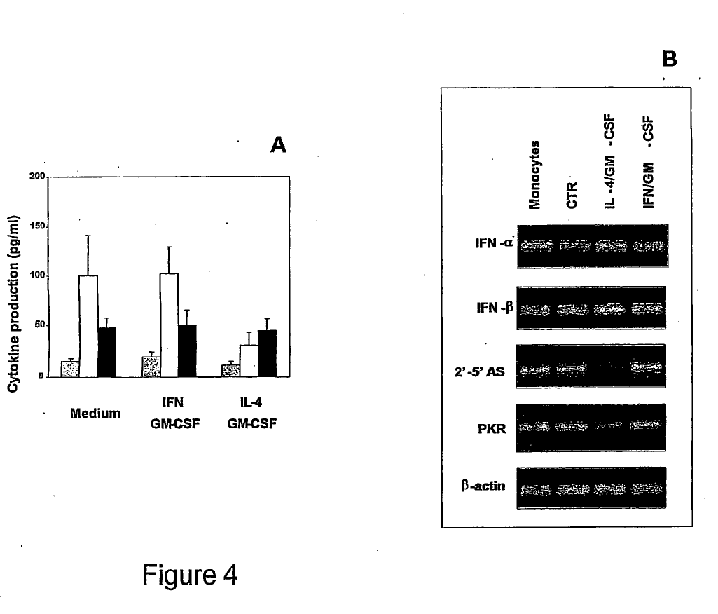

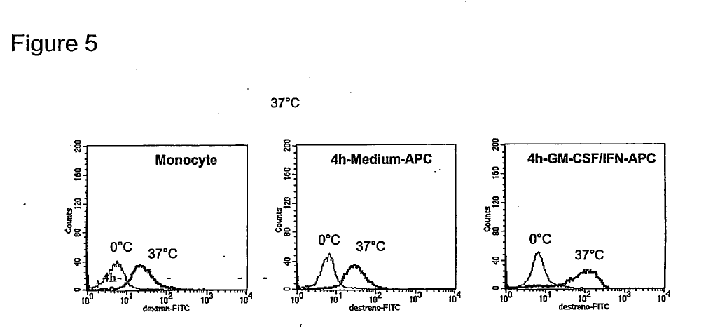

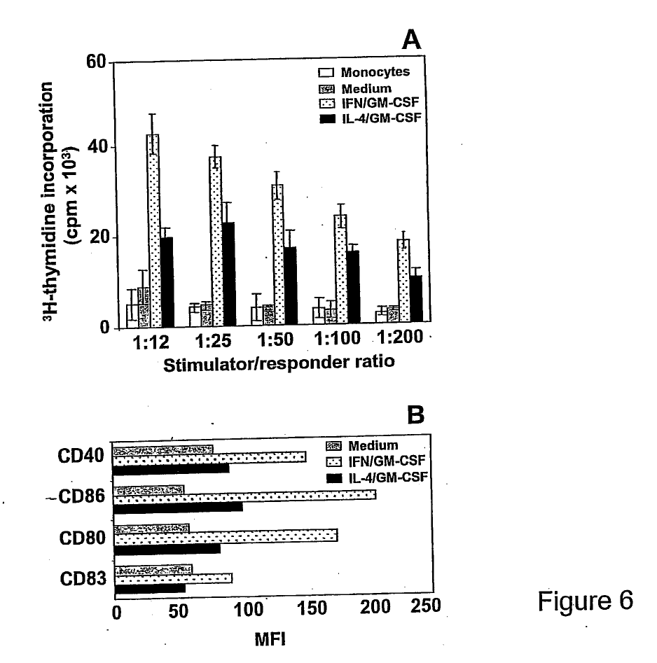

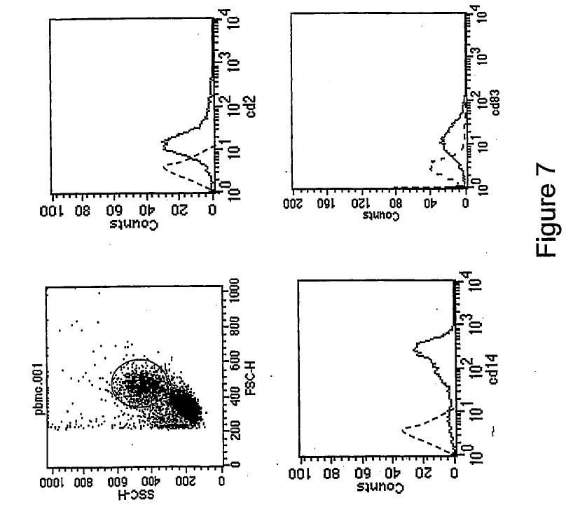

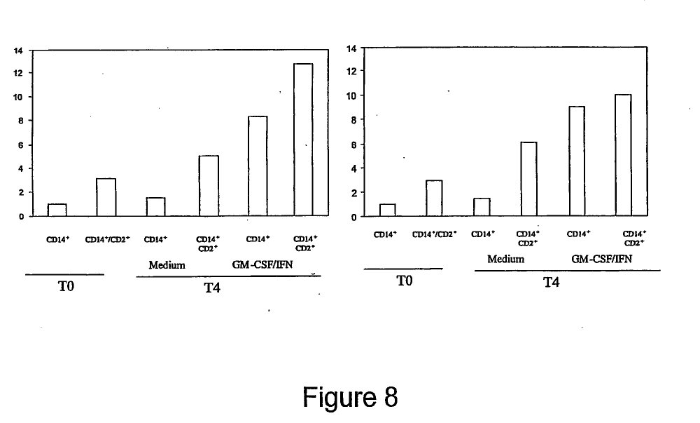

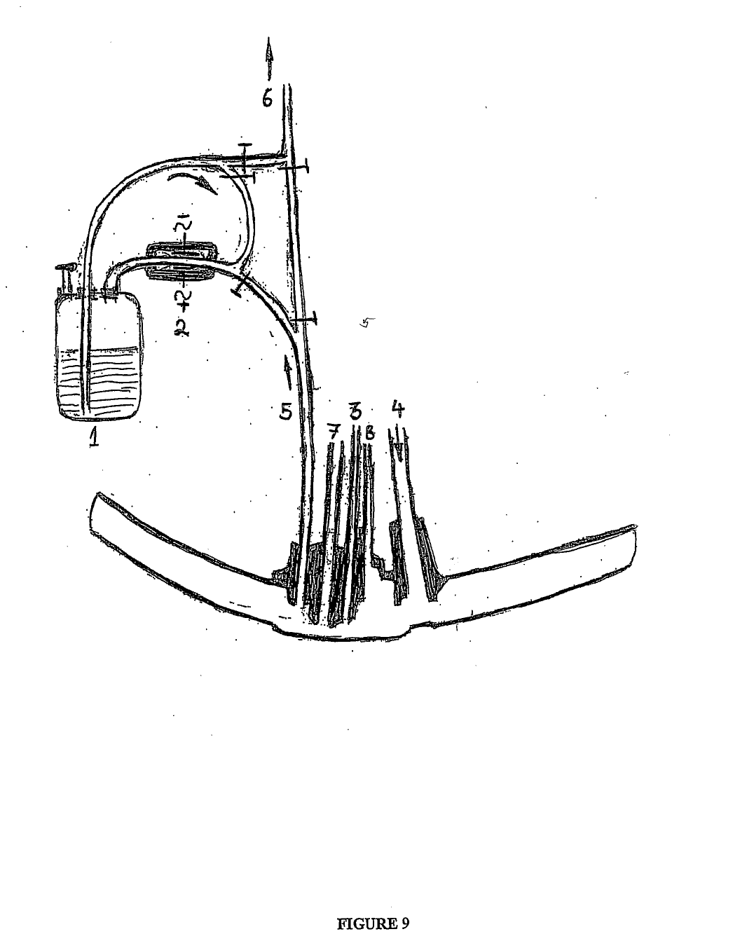

Allogenic mixed lymphocyte reaction (MLR) results are exhibited in FIG. 2C, with proliferation, in counts per minute (y axis), expressed in function of the stimulator/responder ratio (x axis). Freshly separated CD14⁺ cells (•), 4 h-monocyte-derived cells (▪) or monocyte-derived immature dendritic cells (♦) were cultured with allogeneic CD14⁺ T lymphocytes. Proliferation of control lymphocytes was lower than 1,000 cpm. The results shown are the mean ±SE of values from triplicate wells and are representative of three independent experiments. Differences observed between CD14⁺ monocytes and monocyte-derived cells are statistically significant (P<0.04).

FIGS. 3A 3B and 3C: Immunophenotypic analysis of monocytes at early times after treatment with IFN-α/GM-CSF or IL4/GM-CSF, as compared with immunophenotype of immature DCs.

Phenotype of cells recovered after treatment of freshly isolated monocytes with either IFN-α/GM-CSF (FIG. 3A) or IL-4/GM-CSF (FIG. 3B) for 4 or 24 h, or for immature DCs (FIG. 3C) are shown. Dotted histograms represent cells stained with isotype-matched control mAbs. The upper and medium panels of each figure show the expression of CD2 and CD83 on cells recovered after 4 h (left of 3A and 3B) and 24 h (right of 3A and 3B) of treatment, respectively. The results from one representative experiment out of three are shown. The lower histograms of each figure show the expression of co-stimulatory molecules on cells recovered after 4 or 24 h treatment with either IFN-α/GM-CSF or IL-4/GM-CSF, or on monocyte-derived immature DCs. The costimulatory molecule levels are expressed as mean of fluorescence intensity (MFI)±SE of values detected in three independent experiments. The numbers on the bars indicate the percentage ±SE of cells expressing each antigen.

FIGS. 4A and 4B: Secretion of cytokines and expression of mRNA for type I IFN and IFN-induced genes in freshly isolated and cultured monocytes.

Supernatants from monocytes cultured for 4 h in medium alone or medium containing either IFN/GM-CSF or IL-4/GM-CSF were analyzed for the amounts of the different cytokines (FIG. 4A). The x axis represents the amount of cytokine production, in pg/ml. IL-12 p70 amount is indicated with gray bars, TNF-alpha with white bars and IL-1 beta in black bars. The results shown are the mean±SE of values obtained in experiements performed with monocytes from three different donors. Differences in TNF-α levels of untreated and IFN/GM-CSF-treated cultures vs IL-4/GM-CSF-treated cultures are statistically significant (P<0.04).

FIG. 4B shows the RT-PCR analysis of mRNA expression for IFN-α, IFN-β, 2′-5′-AS and PKR in purified monocytes and in 4 h-cultured cells, with medium alone, or monocytes cultured for 4 h in medium alone or medium. The data are representative of one out of three experiments.

FIG. 5: FITC Dextran uptake by freshly isolated monocytes and by monocyte-derived cells after 4 h-culture.

Cells were incubated with FITC-dextran 70 KDa for 30 minutes, either at 37° C. or on ice. The left picture relates to freshly isolated monocytes, middle picture relates to monocyte-derived cells after a 4 hours culture in medium alone, while the right picture relates to cells obtained after a 4 hours culture in the presence of IFN and GM-CSF. In each picture, the darker histogram represent the type of cell studied after incubation at 37° C., while open histogram represents related control cultures incubated on ice (background staining). On each figure, the x axis relates to the number of Dextran-FITC molecules uptaken by the cells, whereas the y axis refers to the counts per cell.

FIGS. 6A and 6B: Antigen presentation ability of freshly isolated monocytes and monocytes cultured with or without cytokines and phenotypical maturation/activation of monocyte-derived cells following co-cultivation with autologous CD4⁺ T lymphocytes.

Freshly isolated monocytes or monocytes cultured for 4 h with or without cytokines were pulsed with inactivated-HIV-1 and then incubated with autologous CD4⁺ T lymphocyte. FIG. 5A shows T lymphocytes proliferation, expressed as thymidine incorporation (y axis) relative to different stimulator/responder ratio (x axis). Proliferation of control CD4⁺ T lymphocytes was lower than 1,000 cpm. The results shown are the mean±SE of values from triplicate wells and are representative of three independent experiments. Differences observed between IFN/GM-CSF-treated cultures and IL-4/GM-CSF-treated cultures are statistically significant (P≦0.05).

FIG. 5B shows phenotypical evolution of cells following co-cultivation with autologous CD4⁺ T lymphocytes.

Monocytes cultured for 4 h with or without cytokines (IFN or IL-4), incubated with autologous CD4⁺ T lymphocytes at a stimulator/responder ratio 1:5. After 4 days, the cells were recovered and analyzed by flow cytometry. Results are expressed as MFW, calculated by subtracting the MFI values of cultures stained with the isotype control mAb from the values obtained by staining with the specific mAbs. The results are representative of one out of three experiments.

FIG. 7: In vitro up-regulation of CD83 expression on the cell membrane of APCs prepared from total PBMCs.

Left and up panel shows the flow cytometric analysis of the cell population obtained after 4 h of exposition of total PBMCs to IFN and GM-CSF. The surrounded points correspond to the selected sub-population analysed for surface expression of the following surface markers: CD2, CD14, CD83. The up and right panel shows the cells surface staining for CD2, the left and low panel shows the staining for CD14 and the right and low panel shows the CD83 staining.

FIG. 8: Stimulation by sorted and unsorted CD2+/CD14+ monocytes and related activated mononuclear APCs of autologous T lymphocytes in the presence of a specific antigen, HIV-1.

FIG. 8 shows the results of autologous T lymphocytes stimulation by different cell populations, for two different blood donors, C and D (respectively left and right panel). Histograms represent 3^{H}-thymidine incorporation, expressed as stimulation index, on y axis, for each cell population. In each panel, the two left histograms correspond to freshly isolated blood monocytes, either after purification of CD14+ cells by immunobeads (CD14+, first left histogram in each panel), or purification of CD14+ cells by immunobeads followed by staining with CD14-FITC and CD2-PE and FACS sorting (CD14+/CD2, second left histogram in each panel). The other four histogram in each panel correspond to activated mononuclear cells prepared from either CD14+ or CD2+/CD14+ monocytes after 4 hours of culture in the presence of culture medium only (two middle histograms) or in the presence of culture medium added with GM-CSF 500 U/ml and IFN 10,000 U/ml (two right histograms). Sorted or cultured cells were pulsed with AT-2-inactivated HIV-1 and cultured in triplicate wells with autologous CD4+ lymphocytes (ratio APC/T 1:50).

FIG. 9: Schematic representation of a kit according to the invention.

The graphic represents a conventional apheresis machine for which the ring concentrating blood mononuclear cells is linked to an additional non adherent plastic bag (Ethylene Vinyl Acetate or Teflon) previously filled with 50 to 500 ml of medium containing cytokine(s) or inducers of interferon synthesis and possibly the antigens to be loaded. After one to five hours of contact, the cells can either be injected directly to the patient's blood stream, or returned for centrifugation to concentrate and/or wash the activated mononuclear cells before re-infusion in autologous plasma to the patient.

In a possible setting, the plastic bag is connected to an electropulsation chamber, allowing the delivery of electric, pulsations to the cell flow, and therefore stressing the mononuclear cells. A single use kit of bags and tubings is provided, allowing sterile connection to the apheresis machine.

1 designates the “cell activation bag” containing medium, cytokines/inducers of interferon synthesis and possibly antigens. 2 designates an optional flow electropulsation chamber. 3 represents a connection for control and possible additions, 4 represents the blood entry, 5 the mononuclear cells entering the cell activation bag. 6, 7 and 8 correspond, respectively, to the flow of activated mononuclear cells, red blood cells and plasma returning to peripheral blood.

Table 1: Cell surface molecules expressed by peripheral blood monocytes upon exposure to IFN/GM-CSF for 4 hours.

Relatively to the different surface markers (left column of the table), surface expression is indicated as the percentage of cells positive for the marker (middle column) and Mean Fluorescence Intensity (MFI, right column), directly related to the mean intensity of fluorescent signal associated to the cells and to the number of said molecules on the cell surface. MFI are arbitrary units, defined as relative fluorescence intensities for each fluorochrome in a cell sample (Becton Dickinson Immunocytometry Systems, Computer Based Training, vol. I, “Flow cytometry and Immunology basics).

| | | |
|---|---|---|
| CD2 | 50 ± 23 | 20 ± 8 |
| CD83 | 49 ± 26 | 24 ± 12 |
| CD14 | 96 ± 3 | 223 ± 24 |
| CD1a | 0 | 0 |
| CD62L | 7 ± 5 | 25 ± 15 |
| CD4 | 88 ± 3 | 38 ± 5 |
| CD40 | 7 ± 6 | 14 ± 10 |
| CD80 | 0 | 0 |
| CD86 | 98 ± 2 | 49 ± 18 |
| CD33 | 96 ± 2 | 130 ± 10 |
| CD25 | 0 | 0 |
| LFA-1 | 95 ± 1 | 1309 ± 59 |
| CD32 | 99 ± 1 | 147 ± 18 |
| CD54 | 96.9 ± 1 | 131 ± 30 |
| CD58 | 94 ± 3 | 130 ± 8 |
| HLA-A, B, C | 97 ± 3 | 158 ± 20 |
| HLA-DR | 98 ± 2 | 600 ± 108 |
| ManR | 3 ± 1 | 21 ± 1 |

Abbreviations Used:
2′-5′-AS, 2′-5′-oligoadenylate-synthetase
APC: antigen presenting cell
AT, aldrithiol
DC, dendritic cell
FITC, Fluorescein IsoThioCyanate
GM-CSF: Granulocyte Monocyte Colony Stimulating Factor.
ManR: Mannose receptor
PE, PhycoBrythrin
PBMC: Peripheral Blood Mononuclear Cells
PKR, double-stranded-RNA-dependent protein kinase;

### EXAMPLES

### Example 1

### Induction of CD83 Expression on the Cell Membrane of Freshly Isolated CD14⁺ Monocytes After a Short-Term Culture: Correlation With the CD2 Expression and With the Acquisition of a Potent MLR Activity

1) Monocyte-Derived Cell Cultures

Monocytes were isolated from heparinized blood of normal donors by Ficoll and Percoll density gradient centrifuigation (Seromed, Berlin, Germany) and immunoselection using a monocyte isolation kit (Miltenyi Biotech, Bergisch Gladbach, Germany). Monocytes were plated at the concentration of 2×10⁶ cells/ml in RPMI 1640 (Gibco BRL, Gaithesburg, Md.) supplemented with 10% FCS (Hyclone Laboratories, Logan, Utah), (with or without the presence of granulocyte-macrophage colony-stimulating factor (GM-CSF; 500 U/ml) and either IL-4 (1,000 U/ml) (R & D Systems, Minneapolis, Minn.) or IFN-α (10,000 U/ml, Alfaferone, alfa-Wasserman, Italy) for 4-24 h). Immature monocyte-derived DCs were obtained by culturing CD14+ monocytes for 6 days in the presence of GM-CSF (500 U/ml) and IL-4 (1,000 U/ml). All reagents were tested for the absence of detectable levels of LPS, by Limulus Amaebocyte lysate assay (Bio-Whittaker, New Jersey, USA).

2) Immunophenotypic Analysis

Cells were washed and resuspended in PBS containing 1% human serum and incubated with fluorochrome-conjugated mAbs for 30 min at 4° C. The following mAbs were used for immunofluorescent staining: anti-CD14, CD80, CD4, CD25, CD54, HLA-DR (Becton Dickinson, San Jose Calif.), CD40, CD86, CD83, CD1a, CD62L, HLA A-B-C, ManR, CD58, LFA-1, CD32 (Pharmingen, San Diego, Calif.), CD2 (clone SFCI3Pt2H9; Beckman Coulter, Fullerton, Calif.), CD33 (Immunotech). Samples were collected and analysed by using a FACSort (Becton Dickinson) and data analysis was performed by CellQuest software (Becton Dickinson). Cells were electronically gated according to light scatter properties, in order to exclude cell debris and contaminating lymphocytes.

3) Statistical Analysis

The correlation between the percentage of starting CD2⁺/CD14⁺ monocytes and the percentage of monocyte-derived CD83⁺ cells recovered after 4 h of culture was analyzed by regression analysis. Statistical analysis was performed using unpaired Student's t-test.

4) Results

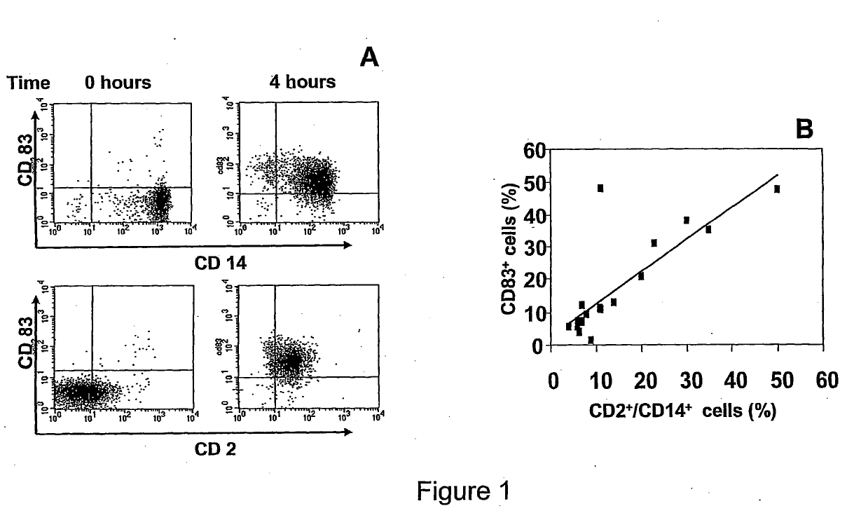

Circulating CD14+ monocytes were purified and cultured in medium without cytokine addition for 4 h; then, the loosely adherent cells were analysed for the expression of CD83, CD14 and CD2 antigens with respect to freshly purified monocytes. Loosely adherent cells are defined as cells differing from monocytes, said monocytes firmly adhering to the plate and needing scraping to be detached. Freshly isolated monocytes did not express CD83, a considerable even though variable percentage of the non adherent cells proved to be CD83⁺ following to their culture (range in 18 donors: 9-93%; median: 42.3%). Notably, these CD83+ cells were also positive for CD14 and CD2 antigens (FIG. 1A). These monocyte-derived CD83⁺/CD14⁺/CD2⁺ cells expressed CD11c, CD13, CD33, CD45RO, CD86 and high levels of major histocompatibility complex class I and class II molecules, while were negative for CD1a and CD62L antigens. In a study with monocytes from 18 donors, the percentage of CD2⁺ monocytes ranged from 4.5% to 50% of the total CD14⁺ circulating monocytes (median: 9%). There was a correlation (r=0.79, P<0.005 by regression analysis) between the percentage, expressed with respect to the initial number of monocytes, of monocyte-derived CD83⁺ cells after 4 h of culture and the percentage of CD2⁺/CD14⁺ cells at the onset of culture (FIG. 1B), suggesting that CD2⁺/CD14⁺ monocytes are the immediate precursors of CD83⁺ cells. More complete data related to activated mononuclear APCs are summarized in table 1. Most of the cells are positive for the surface determinants CD58, CD54 and CD86.

The rapid generation of monocyte-derived CD83⁺ cells suggests that CD83 surface expression could be regulated by post-transcriptional events. The CD83 surface expression in cells cultured in the presence of various metabolic inhibitors added at the beginning of the culture was examined. The up-regulation of CD83 expression on the cell membrane occurring after 4 h of monocyte culture was not significantly inhibited by treatment with the transcriptional inhibitor actinomycin D, whereas it was completely blocked by cyclohexamide or brefeldin A (FIG. 2A), which inhibit protein translation and transport through the Golgi complex, respectively. These results indicated that CD83 expression was not due to “de novo” RNA transcription and that both synthesis and export of the CD83 protein on the cell membrane occurred within a short period of culture. Consistently with these results, remarkable levels of CD83 mRNA were expressed in purified monocytes as well as in cells recovered at 4 h of culture (FIG. 2B). Of interest, the loosely adherent monocyte-derived cells recovered at 4 h of culture proved to be as active as immature monocyte-derived DCs when tested in a typical MLR assay using allogeneic lymphocytes, while, as expected, monocytes poorly stimulated lymphocyte proliferation (FIG. 2C). Notably, when purified CD2⁺ monocytes were used as starting cell population, more than 90% of the cells recovered at 4 h were CD83⁺ and showed a high MLR activity. These results demonstrate that a particular type of CD83⁺/CD2⁺/CD14⁺ cells, highly active as APCs in MLR assays, is generated as early as 4 h after ex vivo incubation of freshly isolated CD14⁺ monocytes.

### Example 2

### Persistence of CD83⁺/CD2⁺ Positive Cells, Secretion of Cytokines Associated with DC Maturation and Expression of IFN-Induced Genes

1) Cytokine Detection

TNF-α, IL-12 and IL-1β production was measured in the culture supernatants by commercial ELISA (R&D Systems, Minneapolis, Minn.). The detection limits of the ELISA were as follows: TNF-α: 4.4 pg/ml, IL-12 p70: 5 pg/ml, IL-1β: 1 pg/ml. ELISAs were performed in triplicate

2) Reverse Transcription-Polymerase Chain Reaction (RT-PCR)

The mRNA from monocyte-derived cultures was extracted by RNAzol B (Tel-Test Inc.) and processed as previously described (13). Samples were amplified with specific primer pairs for CD83 (14), IFN-α, amplifying all 14 known human IFN-α subtype genes, and IFN-β as reported elsewhere (15). Primer sequences for IFN-inducible genes (2′,5′-AS and PKR) were amplified as described elsewhere (16) β-actin RT-PCR was run in parallel to normalize the levels of human RNA in all the samples. All RT-PCR products were in the linear range of amplification.

3) Results

When monocytes were cultured in medium alone for further periods of times (24-48 h), CD83 and CD2 expression was progressively lost along with a decreased cell viability In order to investigate whether cytokines could play a role in the rapid generation/persistence of monocyte-derived CD83⁺ cells, purified monocytes were cultured with either IFN-α or IL-4, the cytokine typically used for generating immature DC from monocytes, in an experimental setting where GM-CSF was constantly included as a monocyte survival factor (FIG. 3).

At 4 h, there was no significant difference in the percentage of CD83+ cells recovered from cultures treated with either IFN-α or IL-4 or with untreated cultures, while the CD2 expression was down-regulated in IL-4-treated cells (FIG. 3): mean+/−SD: 66+/−25% of IFN-treated cells versus 60+/−22% of IL-4-treated cells. At 24 h, no CD2 and CD83 expression was observed in IL-4-treated cultures, whereas IFN-treated cells were still positive for both markers (FIG. 3) (25+/−15% of positive cells). The expression of co-stimulatory molecules was barely detectable (in the case of CD40 and CD86) or undetectable (CD80) in both types of cultures at 4 h. At 24 h, IFN-treated cells expressed higher levels of CD40 and CD80 with respect to the IL-4-treated counterparts (FIG. 3, panels A and B, bottom graphs). Immature DCs, prepared in the presence of IL-4 and GM-CSF, do not express the CD2 and CD83 antigens whereas they express co-stimulatory molecules (FIG. 3C).

To further characterize the differences observed among different monocyte-derived cells, the levels of TNF-α IL-1β and IL-12, pro-inflammatory cytokines known to be associated with DC maturation/activation, were measured in cell supernatants harvested at early time of culture (FIG. 4A). Detectable levels of these cytokines were revealed in all the cultures. However, a marked difference in the extent of TNF-α secretion was observed. In fact, at 4 h, both IFN-treated and untreated cultures secreted higher levels of TFN-α than IL-4-treated cells (FIG. 4A). IL-10 secretion was not detected at either 4 and 24 h, in any cell culture

It was of interest to evaluate the expression of type I IFN genes as well as of genes typically up-regulated by IFN in freshly isolated monocytes with respect to cells cultured for a short time in the presence or absence of cytokines. Considerable levels of mRNA for both IFN-α and IFN-β were detected in freshly isolated monocytes as well as in monocyte-derived cells harvested at 4 h (FIG. 4B). Likewise, there was a clear-cut expression of mRNA of the two IFN-induced genes 2′-5′-oligoadenylate-synthetase (2′-5′-AS) and double-stranded-RNA-dependent protein kinase (PKR) in both freshly isolated monocytes and monocyte-derived cells cultured for 4 h. Notably, the expression of both these IFN-induced genes was lower in IL-4-treated cells (FIG. 4B).

### Example 3

### A Short-Term Exposure of Monocytes to IFN-α Results in the Rapid Generation of Highly Active APCs Capable of Inducing a Primary T Cell Response

I) Antigen Uptake Assay:

Monocytes and cells that were loosely adherent after 4 hours of culture were incubated with FITC-conjugated dextran, 70,000 MW (Molecular Probes, Eugene, Oreg.) at a concentration of 0.1 mg/ml for 30 mn at either 37° C. or on ice (the latter for background staining). The uptake was stopped by adding cold PBS containing 2% FCS and 0.01% NaN3. Cells were washed 3 times and analysed in a FACSort.

2) Results

To evaluate the antigen uptake capacity of the loosely adherent cells recovered after 4 hours of culture in the presence or absence of cytokines, we tested FITC dextran uptake in the cells with respect to monocytes. As shown in FIG. 5, IFN-treated cells that had been cultured for 4 hours exhibited a dextran uptake capability higher than that observed in cells cultured without cytokines for 4 hours, whose phagocytic activity proved to be comparable to that detected in freshly isolated monocytes.

### Example 4

### A Short-Term Exposure of Monocytes to IFN-α Results in the Rapid Generation of Highly Active APCs Capable of Inducing a Primary T Cell Response

1) Mixed Lymphocyte Reaction.

CD4⁺ T lymphocytes were sorted using anti-CD4 conjugated magnetic microbeads (Miltenyi Biotech) and seeded into 96 wells plates (Costar, Cambridge, Mass.) at 10⁵ cells/well. Allogeneic monocyte-derived cells were added (at carious ratios) to each well in triplicate. After 5 days, 1 μCi of methyl-³H-Thymidine (Amersham Pharmacia Biotech AB, Uppsala, Sweden) was added to each well and incubation was continued for additional 18 h. Cells were finally collected by a Mach II Mcell (Tomtec, Amdem, Conn.) harvester and thymidme uptake was quantitated by liquid scintillation counting on 1205 Betaplate (Pharmacia, New Jersey, USA).

2) Primary Response to HIV-1 Antigens

CD4⁺ lymphocytes were purified and seeded into 96 wells plates (Costar) at 10⁵ cells/well. HIV-1 SF162 strain was inactivated by aldrithiol (AT)-2 treatment, following the procedure described elsewhere (17) and stored at −140° C. until use. Loosely adherent cells recovered at 4 h of monocyte cultures were pulsed with AT-2-inactivated HIV-1 (2.4 ng of p24) for 30 min at 37° C. and added to each well in triplicate, at a different ratios. After 7 days, 1 μCi of methye-³H-Thymidine (Amersham) was added to each well for additional 18 hours. Cells were collected and thymidine uptake was quantified as described above. For immuno-phenotypical analysis, monocyte-derived cell-T lymphocyte co-cultures were plated at ratio of 1:5. After 4 days, cells were collected and immunostained for FACS analysis. Analysis was performed by gating on monocyte-derived cells on the basis of the forward and side scatter properties.

3) Results

The phenotypic changes described above were correlated with the possible antigen presenting cell activity, as evaluated by a typical MLR assay using allogeneic lymphocytes as well as by measuring the capability of these cells to induce a primary antigen-specific autologous T cell proliferation. At 4 h of culture, untreated cells as well as cells treated with either IFN/GM-CSF or EL-4/GM-CSF showed a comparable potent MLR activity.

Of interest, when the different cell cultures were tested for their capability to stimulate the proliferation of autologous naive T cells after pulsing with the chemically inactivated HWV-1, a clear-cut difference in their functional activity was observed (FIG. 6A). In fact, at 4 h of culture, IFN-treated cells were more powerful stimulators than cells treated with IL-4/GM-CSF in inducing autologous CD4⁺ T cell proliferation at all the stimulator/responder ratios (P≦0.05). Similar differences between IFN-treated cultures and IL-4-treated cells were observed at 24 h Freshly isolated monocytes as well as untreated cultures did not show any significant capability to stimulate a primary T cell response (FIG. 6A). Notably, when the 4 h-IFN-treated cells were co-cultured with autologous CD4⁺ T cells, a marked up-regulation of the expression of CD80, CD86, CD40 and CD83 was observed at 4 days. The expression of these markers was considerably lower in cultures treated with IL-4 or left untreated (FIG. 6B). Collectively, these results indicate that a short-term treatment of monocytes allows to obtain monocyte-derived APCs capable of efficiently inducing a primary T cell response.

### Example 5

### Induction of CD83 Expression on the Cell Membrane of Total PBMCs After a Short-Term Culture

Total mononuclear cells were collected by apheresis with Cobe Spectra programmed cycle (Cobe, USA) and suspended at 2×10⁶ cells/ml in AIM-V medium containing natural IFNα (10,000 IU/ml) and GM-CSF 500 IU/ml. All reagents were tested for the absence of detectable levels of LPS, by Limulus Amaebocyte lysate assay (Bio-Whittaker, New Jersey, USA).

After 4 hours of contact, the phenotype of the cells was studied as described in example 1. Cells were washed and resuspended in PBS containing 1% human serum and incubated with fluorochrome-conjugated anti-CD2 (clone SFCI3Pt2H9; Beckman Coulter, Fullerton), anti-CD14, anti-CD83 (Pharmingen, San Diego, Calif.) mAb for 30 min at 4° C. (FIG. 7). Samples were collected and analysed by using a FACSort (Becton Dickinson) and data analysis was performed by CellQuest software (Becton Dickinson). Cells were electronically gated according to light scatter properties, in order to exclude cell debris and contaminating lymphocytes.

Cells were analyzed for the expression of CD2, CD14 and CD83 with respect to freshly purified monocytes. A considerable percentage of the loosely adherent cells expressed either CD83 CD2 and CD14 surface antigen.

Cells are irradiated, to prevent autologous T cells proliferation, and seeded for MLR reaction as described in example 1. Activated mononuclear APCs stimulate more efficiently allogenic T lymphocytes than do freshly isolated PBMCs or CD 14+ monocytes.

### Example 6

### Induction of CD83 Expression on the Cell Membrane of Freshly Isolated CD14⁺ Monocytes After a Short-Term Culture in the Absence of Serum

Monocytes are isolated as described in example 1 and plated at the concentration of 2×10⁶ cells/ml in RPMI 1640 (Gibco BRL, Gaithesburg, Md.) in the absence of serum, with or without the presence of granulocyte-macrophage colony-stimulating factor (GM:-CSF; 500 U/ml) and either IL-4 (1,000 U/ml) (R & D Systems, Minneapolis, Minn.), IFN-α (10,000 U/ml, Alfaferone, alfa-Wasserman, Italy) or IL-13 (50 ng/ml, Sanofi-Synthelabo), for 4 to 24 h.

Monocyte-derived cells are washed, resuspended in PBS containing human serum and incubated with fluorochrome-conjugated mAbs as described in example 1, for testing the expression of surface markers, and in particular CD2, CD14, CD83, CD80, CD40, CD86, CD54 and CD58.

### Example 7

### Induction of CD83 Expression on the Cell Membrane of Total PBMCs After a Short-Term Culture

Total mononuclear cells were collected by apheresis with Cobe Spectra programmed cycle (Cobe, USA) and suspended at 2×10⁶ cells/ml in AIM-V medium containing GM-CSF 500 IU/ml and either natural IFNα (10,000 IU/ml) or IL-13 (50 ng/ml, Sanofi-Synthelabo), as described in example 5, for 4 to 24 h.

Recovered monocyte-derived cells are washed, resuspended in PBS containing human serum and incubated with fluorochrome-conjugated mAbs as described in example 1, for testing the expression of surface markers, and in particular CD2, CD14, CD83, CD80, CD40, CD86, CD54 and CD58.

Cells are further characterized and compared to monocyte-derived cells obtained from contacting either total mononuclear cells or purified monocytes with culture medium, supplemented or not with serum, the medium being either alone or containing GM-CSF and either IFN, IL-4 or IL-13. The different types of cells obtained are characterized for their cytokine secretion, particularly TNF-α, capacity of stimulation of allogenic T lymphocytes, by MLR measurement, and capacity of stimulation of autologous T lymphocytes proliferation in the presence of a specific antigen.

### Example 8

### Stimulation of CD8+ T Cells in an Antigen-Specific Way

CD8+ T cells were purified by negative selection using CD8+ T cell isolation kit (Miltenyi Biotec, Paris, France). Activated mononuclear APCs, immature DCs or mature DCs were pulsed for 2 h at 37° C. with 10 micrograms/ml Melan-A peptide and 5 micrograms/ml b2-microglobulin, treated with mitomycin C, and extensively washed. CD8+ T cells (1.5 10⁵ cells/well) were co-cultured with peptide-pulsed autologous APCs or DCs (3 10⁴ cells/well) in 96-well U-bottom plates in Iscove's medium (supplemented with 10% autologous serum, L-arginine, L-asparagine, and L-glutamine) in the presence of 1000 U/ml IL-6 and 5 ng/ml IL-12. On day 7 and 14, DCs were thawed, matured, pulsed with Melan-A peptide and used to restimulate the T cells, in the presence or absence of 20 U/ml IL2 and 10 ng/ml IL-7. Eight T cells microcultures were stimulated for each DC condition and independently tested.

Results:

Activated mononuclear APCs prepared according different condition and immature or mature DCs are pulsed with the analogue Metan-A₂₆₋₃₅(27 L) and used to stimulate autologous CD8+ T cells. After 2 stimulations, CD8+ T cells are tested by IFN-gamma Elispot and in Cr51 release assay. Activated mononuclear APCs are quite efficient in the generation of Melan-A-specific CD8+ T cells. These T cells demonstrated specific cytotoxicity against T2 target cells loaded with Melan-A peptide.

### Example 9

### Treatment of Patients with Chronic Hepatitis B or/and C

Patients with infectious diseases affecting peripheral blood cells (HIV, hepatitis . . . ) are usually excluded from adoptive cellular therapies for safety and regulatory reasons.

In the present intention, those patients are treated during apheresis in a close system, by activating their blood peripheral immune cells in the presence of viral antigens and stimulating cytokines. These mononuclear cells are exposed to IFN α (1000 IU/ml)+GM-CSF (500 IU/ml), HCV antigenic epitopes isolated from virus capside, HBS and HBC antigens at 1 μg/ml in 250 ml RPMI medium for 4 hours. At the end of this contact in non adherent EVA plastic bags connected to the aphaeresis machine, the cells are transferred back in the core of the machine, centrifuged and re-suspended in 50 ml patients plasma for re-infusion. The patients return home after the 4 hours aphaeresis cession which is repeated after 2 weeks and again after 3 months.

Evaluation of this clinical protocol is conducted at months 6. What is observed is a negativation of viral (HBV and/or HCV) load measured by PCR.

### Example 10

### Treatment of Patients with Metastatic Breast Cancer

Patients with metastatic breast cancer after secure ablative surgery of primary tumor received combined chemotherapy and their mononuclear cells were mobilized by injection of GM-CSF.

These patients were then submitted to therapeutic apheresis. The mononuclear cells collected were transferred to a non adherent bag containing AIMV medium IL-18 1 μg/ml plus IL-12 1 μg/ml and oligopolylys -polyhistDNA plasmid coding for HBER2/neu and for MUC-1 antigens. After 3 hours of contact, cells were submitted to a short electropulsation stress. They were kept for one hour at room temperature before washing by centrifugation in the apheresis machine, then re-suspended in 50 ml patients plasma and reinfused to the patient. This treatment is repeated after 1 month. The patients status is monitored by jetscan and clinical evaluation at months 3 and 6.

A progressive decrease in metastatic nodules is seen in lymph nodes and in bones.

### Example 11

### Stimulation by Sorted and Unsorted CD2+/CD14+ Monocytes and Related Activated Mononuclear APCs of Autologous T Lymphocytes in the Presence of a Specific Antigen

After purification of CD14+ cells by immunobeads, monocytes were stained with CD14-FITC (Becton Dickinson) and CD2-PE (Coulter) antibodies and sorted by FACS. Sorted cells were cultured in triplicate wells with autologous CD4+ lymphocytes (ratio APC/T 1:50) or plated at the concentration of 2×10⁶ cells/ml in AIMV (Gibco BRL) with or without the presence of granulocyte-macrophage colony-stimulating factor (GM-CSF; 500 U/ml) and IFN-α (10,000 U/ml, Alfaferone, alfa-Wasserman, Italy) for 4 h. Loosely adherent cells recovered after 4 h of culture of sorted and unsorted monocyte cultures were pulsed with AT-2-inactivated HIV-1 (2.4 ng of p24) for 30 min at 37° C. and added to each well in triplicate (ratio 1:50). After 7 days, 1 μCi of methyl-³H-Thymidine (Amersham) was added to each well for additional 18 h. Cells were collected and thyridine uptake was quantitated by liquid scintillation counting on 1205 Betaplate (Pharmacia, New Jersey, USA).

Results:

FIG. 8 shows the results obtained with either unsorted or sorted cells before and after 4-h of culture with or without IFN/GM-CSF. Purified CD2+/CD14+ monocytes show a higher capability to present antigens than monocytes, notably when sorted CD2+/CD14+ cells were cultured for 4 hours with IFN/GM-CSF, they showed a better activity than the freshly isolated CD2+/CD14+ cell.

CD2+/CD14+ sorted 4-hr-IFN-APCs are even better than the total 4-h-IFN-APC population in terms of antigen presentation.

REFERENCES
1. Steinman, R. M. 1991. The dendritic cell system and its role in immunogenicity. Annu. Rev. Immunol. 9:271-296.
2. Cella, M., F. Sallusto, and A. Lanzavecchia. 1997. Origin, maturation, and antigen-presenting function of dendritic cells. Curr. Opin. Immunol. 9:10-16.
3. Banchereau, J., and R. M. Steinman. 1998. Dendritic cells and the control of immunity. Nature. 392:245-252.
4. Liu, Y. J. 2001. Dendritic cell subsets and lineages, and their functions in innate and adaptive immunity. Cell. 106:259-262.
5. Zhou, L. J., R Schwarting, H. M. Smith, and T. F. Tedder. 1992. A novel cell surface molecule expressed by human interdigitating reticulum cells, Langherans cells and activated lymphocytes is a new member of the immunoglobulin superfamily. J. Immunol. 149:735-740.
6. Scholler, N., M. Hayden-Ledbetter, K. E. Hellstrom, I. Hellstrom, and J. A. Ledbetter. 2001. CD83 is a sialic acid-binding Ig-like lectin (Siglec) adhesion receptor that binds monocytes and a subset of activated CD8⁺ T cells. J. Immunol. 166:3865-3872.
7. Randolph, G. J., S. Beaulieu, S. Lebecque, R. M. Steinman, and W. A. Muller. 1998. Differentiation of monocytes into dendritic cells in a model of transendothelial trafficking. Science. 282:480-483.
8. Santini, S. M., C. Lapenta, M. Logozzi, S. Parlato, M. Spada, T. Di Pucchio, and F. Belardelli. 2000. Type I interferon as a powerful adjuvant for monocyte-derived dendritic cell development and activity in vitro and in Hu-PBL-SCID mice. J. Exp. Med. 191:1777-1788.
9. Sallusto, F., and A. Lanzavecchia. 1994. Efficient presentation of soluble antigen by cultured human dendritic cells is maintained by granulocyte/macrophage colony-stimulating factor plus interleukin 4 and downregulated by tumor necrosis factor alpha. J. Exp. Med. 179:1109-1118.
10. Zhou, L., and T. F. Tedder. 1995. Human blood dendritic cells selectively express CD83, a member of the immunoglobulin superfamily. J. Immunol. 154:3821-3835.
11. Takamizawa, M., A. Rivas, F. Fagnoni, C. Benike, J. Kosek, H. Hyakawa, and E. G. Engleman. 1997. Dendritic cells that process and present nominal antigens to naive T lymphocytes are derived from CD2⁺ precursors. J. Immunol. 158:2134-2142.
12. Crawford, K., D. Gabuzda, V. Pantazopoulos, J. Xu, C. Clement, B. Reinherz, and C. A. Alper. 1999. Circulating CD2⁺ monocytes are dendritic cells. J. Immunol. 163:5920-5928.
13. Bocaccio et al. Identification of a clinical grade maturation factor for dendritic cells. J. Immunother., 2002, 25(1), 88-96.
14. Jeannin et al., OmpA targets dendritic cells, induces their maturation and delivers antigen to the MHC class I presentation pathway. Nat. Immunol. 2000, December 1(6): 502-9.
15. Zelle-Rieser C., Ramoner R., Bartsch G., Thrunher M. A clinically approved oral vaccine against pneumotropic bacteria induces the treminal maturation of CD83+ immunostimulatory T cells. Immunol. Lett. 2001 Feb. 1; 76(1):63-7.

1. A cell population prepared from blood mononuclear cells, said mononuclear cells being peripheral blood mononuclear cells (PBMC) or CD14+ monocytes, said population containing from about 5% to about 50% of activated mononuclear antigen presenting cells (APCs) having the following characteristics:
they express surface markers CD2, CD83 and CD14, MHC class I and MHC class II molecules
they secrete TNF-α
they are able to stimulate allogenic T lymphocytes, as shown by mixed lymphocyte reaction (MLR) measurements.

2. The cell population prepared according to claim 1, characterized in that activated mononuclear APCs are able to stimulate autologous T lymphocytes proliferation in the presence of a specific antigen.

3. Activated mononuclear APCs prepared from blood mononuclear cells and having the following characteristics:
they express surface markers CD2, CD83 and CD14, MHC class I and MHC class II molecules
they secrete more than 50 pg/ml of TNF-α
they are able to stimulate allogenic T lymphocytes, as shown by MLR measurements.

4. Activated mononuclear APCs according to claim 3, characterized in that they express the following surface markers: CD2, CD14, CD83, CD54, CD58, CD86, MHC class I and MHC class II molecules.

5. Activated mononuclear APCs according to claim 3, characterized in that they are able to stimulate autologous T lymphocytes proliferation in the presence of a specific antigen.

6. Activated mononuclear APCs according to claim 3, characterized in that they possess phagocytic properties, as shown by dextran-uptake capability.

7. Activated mononuclear APCs according to claim 3, characterized in that they do not secrete detectable levels of IL-10 and secrete less than 100 pg/ml of IL-12.

8. Activated mononuclear APCs according to claim 3, said activated mononuclear APCs having been loaded with antigenic peptides or proteins, with a cellular extract containing at least one antigen or with nucleic acid molecules.

9. A method for preparing mononuclear Antigen Presenting Cells comprising treating said cells with ligands having receptors on the surface of blood monocytes, of cytokines having receptors on the surface of blood monocytes, of inducers of interferon synthesis by blood mononuclear cells, or of a physical stress, or a combination thereof, as means allowing the preparation from blood mononuclear cells in an appropriate medium, for about one to about five hours and preferably for less than about four hours, of a cell population according to claim 1 or of activated mononuclear APCs.

10. The method, according to claim 9, wherein said ligand is chosen among the group consisting of: cell growth factors, complement polypeptides, muramyl dipeptide analogues, natural and synthetic detoxified endotoxin derivatives, histamine, vitamin D3, arachidonic acid metabolites, aminosulfonic acid derivatives, bacillus Calmette-Guérin and bacterial membrane extracts.

11. The method according to claim 9, wherein said cytokine is type I IFN and is selected from the group consisting of: any natural IFNα, any recombinant species of IFNα, natural or recombinant IFNβ and any synthetic type I IFN.

12. The method according to claim 11, wherein said type I IFN concentration in the medium is in a range of about 100 to about 100,000 IU/ml.

13. The method according to claim 9, wherein said cytokine is chosen among the group consisting of: IFN gamma, IL-12, IL-13, IL-18, GM-CSF, TNFα and TGFβ.

14. The method according to claim 9, wherein said cytokine concentration in the medium is in a range of about 0.01 to about 10 μg/ml.

15. The method according to claim 9, wherein said medium contains type-I interferon, at a concentration of about 10,000 IU/ml, and GM-CSF, at a concentration of about 500 IU/ml.

16. The method according to claim 9, wherein said physical stress consists of one of the following events:
the separation of blood mononuclear cells from the plasma contained in the blood initially containing the mononuclear cells,
the exposure of the blood mononuclear cells to an osmotic change,
the exposure of the blood mononuclear cells to an electrical field or
the exposure of the blood mononuclear cells to a temperature variation of +/−3 to 8° C. from 37° C.

17. A process for preparing, from blood mononuclear cells, a cell population according to claim 1, or activated mononuclear APCs, comprising a step of contacting said mononuclear cells with an appropriate medium, for about one to about five hours and preferably for less than about four hours.

18. The process according to claim 17, characterized in that said medium contains, from the initial stage of the preparation, a component selected from the group consisting of: ligands having receptors on the surface of monocytes, cytokines having receptors on the surface of monocytes and inducers of interferon synthesis by blood mononuclear cells or a combination thereof.

19. The process according to claim 18, wherein said ligand is chosen among the group consisting of: cell growth factors, complement, muramyl dipeptide analogues, natural and synthetic endotoxin derivatives, histamine, vitamin D3, arachidonic acid metabolites, aminosulfonic acid derivatives, bacillus Calmette-Guérin and bacterial membrane extracts.

20. The process according to claim 19, wherein said cytokine is type I IFN and is selected from the group consisting of: any natural IFNα, any recombinant species of IFNα, natural or recombinant IFNβ and any synthetic type I IFN.

21. The process according to claim 20, wherein said concentration of type I IFN in the medium is in a range of about 100 to about 100,000 IU/ml.

22. The process according to claim 18, wherein said cytokine is chosen among the group consisting of: IFN gamma, IL-12, IL-13, IL-18, GM-CSF, TNFα and TGFβ.

23. The process according to claim 22, wherein said concentration of cytokine in the medium is in a range of about 0.01 to about 10 μg/ml and preferably in a range of about 0.1 to about 1 μg/ml.

24. The process according to claim 23, wherein said medium contains type-I interferon, at a concentration of about 10,000 IU/ml, and GM-CSF, at a concentration of about 500 IU/ml.

25. The process for preparing, from blood mononuclear cells, a cell population according to claim 1, or activated mononuclear APCs, comprising a step of exposing said blood mononuclear cells to a physical stress, which can be: the separation of blood mononuclear cells from the plasma contained in the blood initially containg the mononuclear cells, the exposure of the cells to an osmotic change, to an electrical field or to a temperature variation of +/−3 to 8° C. from 37° C.

26. The process according to claim 18, wherein said medium also contains antigenic peptides or proteins, a cellular extract containing at least one antigen or nucleic acid molecules.

27. The process according to claim 18, wherein said process comprises:
a first step of contacting mononuclear cells with an appropriate medium for about one to about five hours and preferably for less than about four hours, for the preparation of activated mononuclear APCs
a second step of contacting said activated mononuclear APCs with a maturation agent.

28. Activated mononuclear APCs such as obtained by a process according to claim 17.

29. A cell population according to claim 1, or activated mononuclear APCs, wherein activated mononuclear APCs are kept under a frozen form, in an appropriate cryo-preservative solution.

30. (canceled)

31. (canceled)

32. A kit for preparing, from blood mononuclear cells, a cell population according to claim 1 or activated mononuclear APCs, in a close system allowing the exclusion of any conventional ex-vivo culture step and comprising at least:
single use elements necessary for the culture and the washings of the cells, including bag(s), culture medium, buffers and connecting tube(s), including connecting tube(s) to an apheresis machine,
possibly a composition comprising type I IFN and compatible additives,
possibly a composition comprising a cytokine and compatible additives,
possibly a composition comprising a ligand having receptors on the surface of blood monocytes and compatible additives,
possibly a composition comprising a cell growth factor and compatible additives,
possibly a composition comprising at least one antigen, or nucleic acids coding for at least one antigen, to which an immune response is of interest.

33. A pharmaceutical composition or a vaccine comprising, as active principle, activated mononuclear APCs according to claim 3, together with a pharmaceutically acceptable carrier vehicle or an auxiliary agent, in an amount of about 10⁵ to about 10¹⁰ of said cells per dose administered.

34. A pharmaceutical composition or a vaccine containing, as an adjuvant of an active principle, activated mononuclear APCs according to claim 3, in an amount of about 10⁵ to about 10¹⁰ of said cells per dose administered.

35. A method for treating an infectious or neoplastic disease in a patient, comprising administering an effective amount of a cell population according to claim 1 to said patient in need thereof.

36. A method for treating an infectious or neoplastic disease in a patient, comprising administering an effective amount of activated mononuclear APCs according to claim 3 to said patient in need thereof.
